(19) United States
(12) Patent Application Publication
(10) Pub. No.: US 2022/0073900 A1
(43) Pub. Date: Mar. 10, 2022

(54) CELL ANALYSIS SYSTEMS WITH CELL ENCAPSULATION

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 17/417,312

(22) PCT Filed: Feb. 1, 2019
(86) PCT No.: PCT/US2019/016376

§ 371 Date: Jun. 22, 2021

(51) Int. Cl. C12N 11/04; C12Q 1/6806; B01L 3/00

(52) U.S. Cl. CPC C12N 11/04; C12Q 1/6806; B01L 2200/16; B01L 2200/0652; B01L 2300/0663; B01L 3/502761

(57) ABSTRACT

In one example in accordance with the present disclosure, a method is described. The method includes receiving, in a microfluidic channel, serially fed cells to be encapsulated. Each cell is individually lysed and a lysate of each cell is transported to a downstream analysis device. The lysate of an individual cell is encapsulated with an encapsulating fluid.

### BACKGROUND

In analytic chemistry, scientists use instruments to separate, identify, and quantify matter. Cell lysis is a process of rupturing the cell membrane to extract intracellular components for purposes such as purifying the components, retrieving deoxyribonucleic acid (DNA), ribonucleic acid (RNA), proteins, polypeptides, metabolites, or other small molecules contained therein, and analyzing the components for genetic and/or disease characteristics. Cell lysis bursts a cell membrane and frees the inner components. The fluid resulting from the bursting of the cell is referred to as lysate.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a flow chart of a method of cell encapsulation, according to an example of the principles described herein.

FIG. 2 is a block diagram of a cell analysis device with cell encapsulation, according to an example of the principles described herein.

FIG. 3 is a block diagram of a cell analysis system with cell encapsulation, according to an example of the principles described herein.

FIGS. 4A -4D are diagrams of a cell analysis device with cell encapsulation, according to another example of the principles described herein.

FIG. 5 is a flow chart of a method of cell encapsulation, according to another example of the principles described herein.

FIG. 6 is a diagram of a cell analysis device with cell encapsulation, according to another example of the principles described herein.

FIG. 7 is a diagram of a cell analysis device with cell encapsulation, according to another example of the principles described herein.

FIG. 8 is a diagram of a cell analysis device with cell encapsulation, according to another example of the principles described herein.

FIG. 9 is a diagram of a cell analysis device with cell encapsulation, according to another example of the principles described herein.

FIG. 10 is a diagram of a cell analysis device with cell encapsulation, according to another example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

In cellular analytics, a scientist uses instruments to separate, identify, and quantify matter. A wealth of information can be collected from a cellular sample.

For example, the mechanical properties of the cell membrane and even more specifically information relating to the mechanical breakdown of the cell membrane can provide insight to the characteristics and state of a cellular sample. For example, in some cases the physical characteristics of a particular cell can be used to classify and/or differentiate the particular cell from other cells. In another example, changes to the physical characteristics of a cell can be used to determine a state of the cell.

The intracellular components of the cell also provide valuable information about a cell. Cell lysis is a process of extracting intracellular components, or lysate, from a cell. During lysis, the intracellular components are extracted for purposes such as purifying the components, retrieving DNA and RNA proteins, polypeptides, metabolites, and small molecules or other components therein, and analyzing the components for genetic and/or disease characteristics. The study and analysis of the lysate of a cell provides information used to characterize and analyze a cell. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others.

While cellular analytics is useful, refinements to the operation may yield more detailed analysis results. For example, in general it may be difficult to obtain a correlation between 1) the mechanical and chemical properties of a cell and 2) the genetic information of the cell. That is, a user cannot simultaneously get mechanical and genetic information from a single sample. To get both genomic and mechanical information, two different samples would be used. However, as the different samples may have different properties, any correlation between the separately collected genomic and mechanical information would rely on a similarity between the two samples, which similarity may not exist or may be tenuous.

Accordingly, a scientist may have to pick from between the two pieces of information (e.g., mechanical and genomic), which they would like to collect. It may be more desirable to obtain the genomic information from the cell as it provides more information. However as described above, the mechanical properties of a cell also provide valuable information. For example, lysis information allows a user to infer cell mechanical properties which may indicate to the user the state of the cell, i.e., dead/living, diseased/healthy.

Moreover, in cellular analytics it may be desirable to know the correlation between a phenotype and a genotype of a cell. Information about this correlation ay lead to a better understanding of chemical signaling pathways within the cell. Knowing the chemical signaling pathways allows for a greater understanding of cell function and response to stimuli. For example, a correlation between genomic information and a cells susceptibility to lysis may allow a prediction of lytic antibiotic resistance of a cell based on the cells' genetic information. Disease pathology is a specific example as mechanical properties play a particular role in disease pathology. For example, the elasticity (mechanical property) of a circulating tumor cell may be a determining factor of the cell's metastatic potential and therefore may be an indicator of cancerous cells. In this example, the genetic information collected form a sample indicates what mutations are activated in the cell and may indicate which pathways are up or down regulated. From the genetic and mechanical information, a medical professional may determine which chemotherapy to prescribe as the role of many chemotherapeutics is to affect these pathways. As yet another example, malaria, which is a parasitic infection of red blood cells that changes a stiffness (mechanical property) of the red blood cells and changes the transportation of these cells through the circulatory system. By obtaining the genetic information at the same time, a scientist may determine a type of parasite (there are many malarial parasites for example) that are affecting the patient. With such detailed solutions, a more specific anti-malarial process may be followed. Accordingly, both pieces of information, i.e., mechanical properties and genetic information, for a cell are valuable and useful in analytic chemistry.

Still further, many cell populations are heterogeneous, meaning each cell in a population may be different from others and may have different responses and characteristics. That is, a particular sample, such as a blood sample, may include a number of different kinds of cells, each to perform different function and different in its physical and chemical makeup. This heterogeneity of a sample is a building block of the foundations of sustainable life. For example, the different cells in blood allow the blood to sustain human life. Accordingly, when a cell sample is analyzed, it may be desirable to separate the lysate from each cell in order to analyze the lysate without the effect of lysates from disparate cells.

Thus, while cells may have an inherent variability that contributes to the overall behavior of the cell sample, it may be desirable to analyze cells individually. To obtain nucleic acid information from cells, cells may be positioned into physical wells. In some cases, multiple cells end up in a single well. A lysis operation is performed in these wells. However, following these operations, the nucleic acids are fairly dilute as the well is much larger than the cell. Moreover, the cells are mixed with protein and debris contaminants. If there are multiple cells in a well, the lysates of these cells are also mixed in the well.

In another example, individual cells may be placed into wells. However, such a process is time intensive and is generally performed on a small number of cells. Thus, throughput is low. Moreover, such a single well still demands that a scientist place the cells in a well exposing the cell to potential contaminants.

Accordingly, either cells are mixed and the resulting lysates are also mixed, thus introducing error into any downstream analysis, or only small numbers of cells may be analyzed, regardless of the fact that the total number of cells in a population is quite large.

The present specification enhances the multi-modal analysis of a single cell by isolating the lysate from the lysates of other cells. Thus, a “clean” analysis of the cell can be performed where any results that are obtained can be properly mapped to that cell, without any variability and/or error that may be introduced by the lysate of the cell mixing with the lysate of other cells. That is, the present specification describes a system for lysing single cells in a feedback-controlled manner, extracting cell components including nucleic acid content, and encapsulating the nucleic acid content to be used in downstream analysis. Specific examples of downstream analysis include polymerase chain reaction (PCR) operations and DNA sequencing.

Specifically, the present method and system lyse large numbers of single cells and capture the contents of a particular cell in a liquid capsule so that the contents of the capsule can be analyzed downstream. In general, the system includes a lysing chamber that receives cells to be lysed. The system also includes an encapsulation mechanism. In one example, the encapsulation is an off-chip mechanism and in another example, the encapsulation mechanism is on-chip, that is it is on/in the same substrate as the lysis device. For off-chip encapsulation, the lysate, which is aqueous, is dispensed as a drop onto an oil, which may be a film on top of an aqueous buffer. As the aqueous lysate drop penetrates the oil film, the film envelopes the drop. As the drop sinks to the bottom, a water-oil emulsion is formed, which emulsion is stabilized by dispersants.

For on-chip encapsulation, the aqueous lysate is passed along a microfluidic channel where encapsulating fluid inlets inject encapsulating fluid into the microfluidic channel. Accordingly, in this example alternating oil-aqueous slugs are formed, wherein each slug is a separate lysate. The encapsulated droplets are then dispensed to be further analyzed downstream.

Specifically, the present specification describes a cell encapsulation method. According to the method, serially fed cells to be encapsulated are received in a microfluidic channel. Each cell is individually lysed and a lysate of each cell is transported to a downstream analysis device. The lysate of an individual cell is encapsulated with an encapsulating fluid.

The present specification also describes a cell analysis device. The cell analysis device includes a microfluidic channel to serially transport cells along a flow path. A lysis device of the cell analysis device is along the microfluidic channel and ruptures a cell membrane of a cell. The cell analysis device also includes a number of encapsulation inlets to deliver a quantity of an encapsulating fluid into the microfluidic channel to encapsulate a lysate of the cell.

The present specification also describes a cell analysis system. The cell analysis system includes at least one cell analysis device. Each cell analysis device includes a microfluidic channel to serially feed individual cells from a volume of cells into a lysing chamber. At least one feedback-controlled lysing element is disposed in the lysing chamber to agitate a cell. Each cell analysis device also includes a number of encapsulation inlets to deliver a quantity of an encapsulating fluid into the microfluidic channel to encapsulate a lysate of each cell. The cell analysis system also includes a controller to analyze the cell. The controller includes 1) a lysate analyzer to analyze properties of a lysate of the cell and 2) a rupture analyzer to analyze parameters of an agitation when a cell membrane ruptures.

In summary, such a cell analysis device, system, and method 1) allow single cell analysis of a sample; 2) allow combined cell analysis, i.e., a genetic analysis and a mechanical property analysis; 3) can be integrated onto a lab-on-a-chip; 4) are scalable and can be parallelized for high throughput, 5) are low cost and effective; 6) are automated; 7) preserve lysate content from contamination by other lysates and other components such as protein and debris which may be in the carrier fluid and RNAase and DNAase which may have been used to lyse the cell; 8) reduce dilution of lysate contents thus improving downstream analysis; and 9) reduce the effect of potential user error. However, the devices disclosed herein may address other matters and deficiencies in a number of technical areas.

As used in the present specification and in the appended claims, the term “cell membrane” refers to any enclosing structure of a cell, organelle, or other cellular particle.

Further, as used in the present specification and in the appended claims, the term “agitation cycle” refers to a period when a cell is exposed to the operations of a lysing element. For example, an agitation cycle may refer to each time a cell is looped past a single lysing element. In another example, a cell passes through an agitation cycle each time it passes by a lysing element in a string of multiple lysing elements.

Even further, as used in the present specification and in the appended claims, the term “rupture threshold” refers to the amount of stress that a cell can withstand before rupturing. In other words, the rupture threshold is the threshold at which the cell ruptures. The rupture threshold may be determined based on any number of factors including a number of agitation cycles a cell is exposed to and the intensity of the agitation cycles.

Yet further, as used in the present specification and in the appended claims, the term “parameters” refers to the operating conditions in a particular agitation cycle. For example, a “parameter” may refer to a type of lysing element and/or a lysing strength. For example, agitation parameters for an agitation cycle may include whether a lysing element is a thermal inkjet resistor, a piezo-electric device, or an ultrasonic transducer. Agitation parameters also refer to the operating conditions of the particular lysing element. For example, the parameters of an ultrasonic transducer may refer to the frequency, amplitude, and/or phase of ultrasonic waves. The parameters of the thermal inkjet resistor and piezo-electric device may refer to the size of the element and/or the voltage applied to the element.

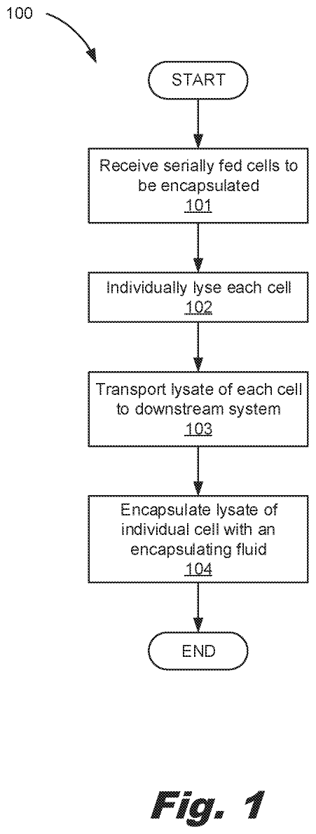

FIG. 1 is a flow chart of a method (100) of cell encapsulation, according to an example of the principles described herein. In the method (100), serially fed cells to be encapsulated are received (block 101) in a microfluidic channel. That is, each cell within the sample may be received (block 101) one at a time. In some examples, each cell analysis device includes a microfluidic channel that gates introduction of one cell at a time into the cell analysis device for lysing and encapsulation. Such single-file, or serial, inlet of cells facilitates an individual processing of cells. Accordingly, rather than lysing a group of cells and hoping that each cell is lysed, individual cells can be lysed, thus providing greater control over downstream cell analysis. By individually encapsulating each cell, more relevant information regarding the analyzed cell is possible as sources of variation and error such as mixing and diffusion of lysates are avoided. Such a serial, single-file introduction of cells into the system may be facilitated by microfluidic channels having a cross-sectional area size on the order of the cell diameter.

Each cell is individually lysed (block 102). That is, the cells pass in single-file fashion to a lysing chamber where lysis occurs. In general, lysis refers to the agitation of a cell with the objective of rupturing a cell membrane. As a cell membrane is ruptured, the inner components are released. The fluid containing the inner components is referred to as lysate. The contents of the cellular particle can then be analyzed by a downstream system. The point at which a cell membrane breaks down may be referred to as a rupture threshold and may provide valuable information about a particular cell as described above.

In some examples, the lysis operation (block 102) is feedback-controlled. That is, in some cases, a lysing element may not rupture a cell membrane. For example, the cell membrane may be robust against a particular intensity of agitation. Without feedback-controlled lysis, the cell may leave the lysing chamber intact. Outputting an intact cell when a lysed cell is desired and/or expected, may result in skewed results. Accordingly, when a sensor indicates that, despite the operations of the feedback-controlled lysing element, the cell membrane has not ruptured, a controller may return the cell to be under the influence of the feedback-controlled lysing element.

In this example, a second lysing cycle, either at the same intensity or an increased intensity, may be executed such that the cell may be ruptured. That is in some examples, agitation intensity may be incrementally adjusted until the cell membrane ruptures. As specific examples, the energy applied to a thermal inkjet resistor may increase or the intensity of ultrasonic waves may increase with each agitation cycle.

Once lysed, the lysate, or internal components of a cell, is transported (block 103) to a downstream analysis device. The transportation (block 103) operation may include a variety of mechanisms. For example, the lysate may be transported through a single, or network, of microfluidic channels to subsequent analysis devices.

In another example, the lysate is ejected for analysis by the downstream analysis device. In this example, the cell analysis device includes an ejector to expel the lysate from the substrate on which the lysis chamber is formed. Put another way, in some examples, the downstream analysis device may be formed on the same substrate as the lysis chamber and in other examples, the downstream analysis device may be on a separate structure.

The downstream analysis device may be of any type. For example, the downstream analysis device may perform a subsequent lysing operation to further break down the components of the cell. In yet another example, the downstream analysis device may be a microarray, or a titration plate. As yet another example, the downstream analysis device may perform a polymerase chain reaction (PCR) on the cell lysate.

In either case, i.e., transportation (block 103) via microfluidic channel on the same structure or transportation (block 103) via ejection to another structure, the lysate of an individual cell is encapsulated (block 104) with an encapsulating fluid. As described above, following lysis, a lysate may diffuse and mix with the lysate of other cells and/or the carrier fluid of the cell. This mixing can obfuscate the results achieved by analysis of the lysate. By encapsulating (block 104) the lysate, the nucleic acid of individual cells may be separated and isolated from the carrier fluid and the lysate of other cells thus enhancing the accuracy and repeatability of analytic operations. Thus, a particular cell lysate can be individually analyzed in a clean environment free from contamination by the carrier fluid and/or other lysates.

The encapsulating fluid may be of a variety of types. In general, the encapsulating fluid is immiscible with the lysate. That is, a lysate may be aqueous. Accordingly, the encapsulating fluid may be an oil that is immiscible with the aqueous-based lysate. In one specific example, the oil in the encapsulating fluid may be hexa-methyl di-siloxane. Other examples of encapsulating fluids include hexadecane, sodium dodecyl sulfate, tetradecane, octadecane, dodecane, polyphenyl methylsiloxane, and mineral oil. While specific reference is made to a few specific encapsulating fluids, other encapsulating fluids may also be used.

In some examples, the encapsulating fluid includes a surfactant mixed with the oil. A surfactant decreases surface tension and stabilizes an interface between molecules that would not otherwise mix. That is, an oil-based encapsulating fluid may not adhere to the aqueous-based lysate. However, a surfactant facilitates this adhesion. Specifically, the surfactant may be amphiphilic meaning it has a hydrophobic component and a hydrophilic component. The hydrophilic heads may be in contact with the aqueous lysate while the hydrophobic tails may be in contact with the oil in the encapsulating fluid. Thus, the surfactant enables the lysate to be enclosed and isolated with an oil-based encapsulating fluid. That is, the surfactant provides for a stable encapsulated lysate that won't break down and that will therefore remain encapsulated. The surfactant may be of a variety of types. For example, the surfactant may be polyethylene glycol p-(1,1,3,3-tetramethylbutyl)-phenyl ether, cetyl PEG/PPG-10-1 dimethicone, cetyl polyethelyne glycol, polypropolyne glucol-10/1 dimethicone, sorbitane monooleate, and sodium deodecyl sulfate.

Once encapsulated, the lysate can then be used in different downstream operations such as digital drop PCR or other droplet-wise cellular analysis. Thus, the present method (100) provides for the lysing of individual cells and for the encapsulation of individual cells. Accordingly, entire cellular analytic operations can be carried out on a per-cell basis, rather than performing cellular analytics on groups of cells.

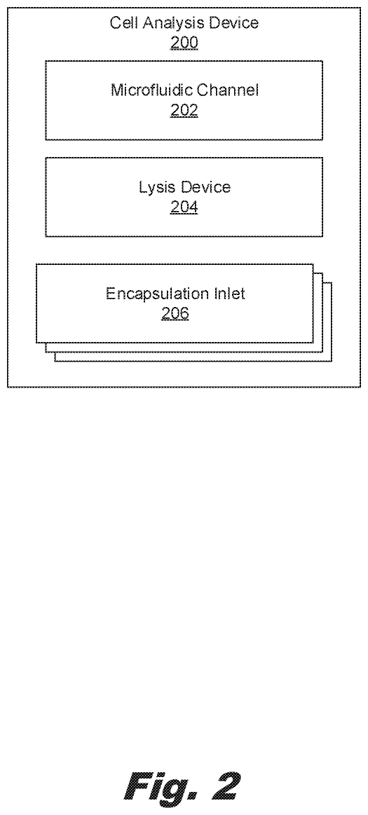

FIG. 2 is a block diagram of a cell analysis device (200), according to an example of the principles described herein. In some examples, the cell analysis device (200) is part of a single integrated system that is multi-functional. Such a system combines several laboratory functions on a single integrated circuit which may be disposed on a silicon wafer. Such systems may be a few square millimeters to a few square centimeters, and provide efficient small-scale fluid analysis functionality.

In other words, the components, i.e., the microfluidic channel (202), lysis device (204), and encapsulation inlets (206) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The microfluidic channel (202) serially transports cells along a flow path. That is, the microfluidic channel (202) is the conduit through which the cells flow. In some examples, environmental conditions direct the flow. For example, the cell reservoir from which the cells originate may be disposed above the cell analysis device (200) such that the effects of gravity draw fluid along the flow path. In other examples, flow may be induced. That is, fluid transport devices such as pumps may push fluid along the flow path.

The microfluidic channel (202) passes the cells in individual fashion along the flow path. That is, the cell analysis device (200) of the present specification describes a per-cell lysis and encapsulation. Such a serial, single-file introduction of cells along the flow path may be facilitated by microfluidic channels (202) having a cross-sectional area size on the order of the cell diameter.

The cell analysis device (200) also includes a lysis device (204) along the microfluidic channel (202) to rupture a cell membrane of the cell. As described above, the lysis device (204) agitates a cell with the objective of rupturing a cell membrane. The lysis device (204) includes a lysing chamber where lysing occurs. In some examples, the lysing chamber may be no more than 100 times a volume of a cell to be lysed. In other examples, the lysing chamber may have a cross-sectional size comparable with the cell size and in some cases smaller than the cell so as to deform the cell before or during the rupturing of the cell membrane. That is, the lysing chamber may be a microfluidic structure. Thus, lysing operations can be performed on a single cell and that cell's particular properties may be analyzed and processed.

In some examples, the lysis operation may be feedback-controlled. Feedback-controlled lysis provides a quality control check over a lysing operation. In this example, the lysing chamber includes a sensor to determine when a cell has ruptured, and to return the cell to within range of the lysing element in the case the cell has not ruptured. That is, the sensor detects a change in the cell based on an agitation of the cell by the at least one lysing element. If no change is detected, the cell is kept in, or returned to, the lysing chamber for another agitation cycle. Accordingly, rather than activating the lysing element and hoping that lysing occurs, a feedback-controlled lysing element includes a sensor to ensure lysing occurs prior to further processing of the lysate.

In some examples, a controller gradually increases the intensity of agitation such that it can be precisely determined at what stress level a particular cell ruptures. Increasing the agitation intensity may include increasing the intensity of the lysing element and/or by increasing a count of how many exposures the cell has to the lysing element. For example, a lysing element intensity may not change, but the cell may be passed by the lysing element multiple times until cell rupture occurs. In another example, a lysing element intensity increases and the cell may be passed by the lysing element multiple times until cell rupture occurs.

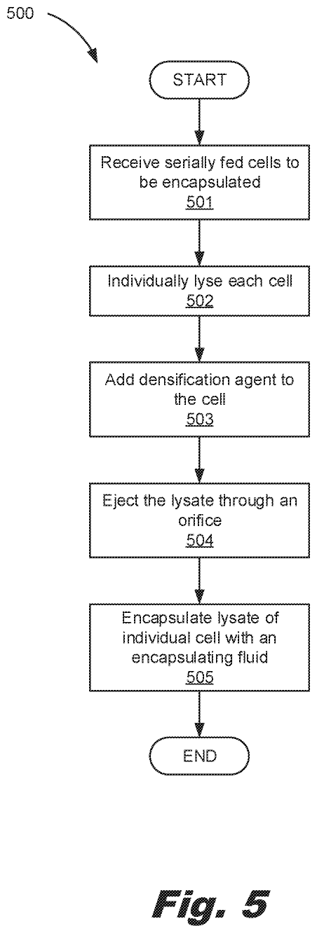

The cell analysis device (200) in this example, includes a number of encapsulation inlets (206) to deliver a quantity of an encapsulating fluid into the microfluidic channel (202) to encapsulate a lysate of the cell. In this example, the encapsulation mechanism is disposed in/on the same substrate as the lysis device (204). An example of this configuration is depicted in at least FIG. 5.

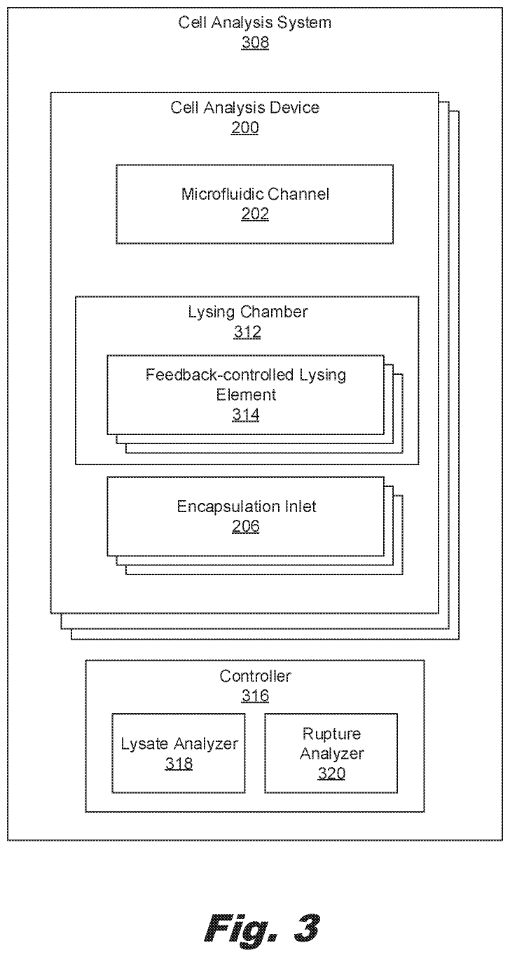

FIG. 3 is a block diagram of a cell analysis system (308), according to an example of the principles described herein. In some examples, the cell analysis system (308) is part of a lab-on-a-chip device. A lab-on-a-chip device combines several laboratory functions on a single integrated circuit which may be disposed on a silicon wafer. Such lab-on-a-chip devices may be a few square millimeters to a few square centimeters, and provide efficient small-scale fluid analysis functionality.

In other words, the components, i.e., the cell analysis device(s) (200), microfluidic channel(s) (202), lysis device (FIG. 2, 204) which includes the lysing chamber (312) and feedback-controlled lysing elements (314), and encapsulation inlet(s) (206) may be microfluidic structures. A microfluidic structure is a structure of sufficiently small size (e.g., of nanometer sized scale, micrometer sized scale, millimeter sized scale, etc.) to facilitate conveyance of small volumes of fluid (e.g., picoliter scale, nanoliter scale, microliter scale, milliliter scale, etc.).

The cell analysis system (308) include at least one cell analysis device (200). In some examples, each component that makes up the cell analysis device (200) is disposed on a single substrate. Thus, each operation may be carried out on a single substrate. That is, the present cell analysis system (308) facilitates the complete analysis of a cell, at a single cell resolution, on a single physical structure. The substrate may be formed of any material including plastic and silicon, such as in a printed circuit board.

In some examples, a single cell analysis device (200) is used in the cell analysis system (308). However, the cell analysis system (308) may include multiple cell analysis devices (200), each to analyze an individual cell. In this example, the multiple cell analysis devices (200) may be in parallel. The multiple parallel cell analysis devices (200) facilitate the processing of more cells. For example, as described above, each cell analysis device (200) analyzes a single cell at a time. Accordingly, multiple parallel cell analysis devices (200) allow multiple cells to be analyzed at the same time, rather than analyzing a single cell at a time.

As described above, each cell analysis device (200) includes various components to perform cell analysis on a per-cell basis. Specifically, the cell analysis device (200) includes a microfluidic channel (202) to serially feed individual cells from a volume of cells into a lysing chamber (312).

At least one feedback-controlled lysing element (314) is disposed in the lysing chamber (312) to agitate the cell. The feedback-controlled lysing element (314) may implement any number of agitation mechanisms, including shearing, ball milling, pestle grinding, and using rotating blades to grind the membranes. Other examples of agitation mechanisms include localized heating and shearing by constriction. In another example, repeated cycles of freezing and thawing can disrupt cells through ice crystal formation. Solution-based lysis is yet another example. In these examples, the osmotic pressure in the cellular particle could be increased or decreased to collapse the cell membrane or to cause the membrane to burst. As yet another example, the cells may be forced through a narrow space, thereby shearing the cell membranes.

In one example, the feedback-controlled lysing element (314) is a thermal inkjet heating resistor disposed within the lysing chamber (312). In this example, the thermal inkjet resistor heats up in response to an applied current. As the resistor heats up, a portion of the fluid in the chamber vaporizes to generate a bubble. This bubble generates a pressure and shear spike which ruptures the cell membrane.

In another example, the feedback-controlled lysing element (314) may be a piezoelectric device. As a voltage is applied, the piezoelectric device changes shape which generates a pressure pulse in the chamber that generates a pressure and shear spike which ruptures the cell membrane.

In yet another example, the feedback-controlled lysing element (314) may be a non-reversible electroporation electrode that forms nano-scale pores on the cell membrane. These pores grow and envelope the entire cell membrane leading to membrane lysis. In yet another example, the feedback-controlled lysing element (314) is an ultrasonic transducer that generates high energy sonic waves. These high energy waves may travel through the wall of the chamber to shear the cells disposed therein.

The different types of feedback-controlled lysing elements (314) each may exhibit a different agitation mechanism. For example, the agitation mechanism of an ultrasonic transducer is the ultrasonic waves that are emitted and that shear the cells. The agitation mechanism of the thermal inkjet heating resistor is the vapor bubble that is generated and ruptures the cell membrane. The agitation mechanism of the piezo-electric device is the pressure wave that is generated during deformation of the piezo-electric device, which pressure wave shears the cell membrane. While particular examples of feedback-controlled lysing elements (314) have been described herein, a variety of feedback-controlled lysing element (314) types may be implemented in accordance with the principles described herein.

Also disposed within the lysing chamber (312) is a sensor used to determine whether the cell membrane was ruptured. The sensor provides the feedback that controls operation of the feedback-controlled lysing element (314). The sensor may take many forms. For example, the sensor may be an optical scatter sensor that determines cell rupture based on a scatter of reflected energy waves. The sensor may be an optical fluorescence sensor that detects cell rupture based on the detection of certain fluorescent markers. In other examples, the sensor may be an optical bright field sensing system, an optical dark field sensing system, or a thermal property sensor.

In one particular example, the sensor is an impedance sensor. Specifically, the sensor may include at least one pair of electrodes spaced apart from one another by a gap. These electrodes detect a level of conductivity within the gap. That is, incoming cells to a lysing chamber (312), and the solution in which they are contained, have a predetermined electrical conductivity. Any change to the contents within the lysing chamber (312) will effectively change the electrical conductivity within the lysing chamber (312). Specifically, as the cells are ruptured and the nucleic acid pours out, the conductivity would increase. To measure the conductivity, a resistance of solution between electrodes of the impedance sensor is measured and a conductivity determined therefrom. In some examples, a single pair of electrodes are used, with one electrode plate placed at either end of the lysing chamber (312). In another example, multiple pair of electrodes are used. For example, one pair of electrode plates could be placed at the inlet and another pair of electrode plates placed at the outlet.

Thus, in summary, the sensor can determine when a cell membrane has been ruptured. If a cell has been ruptured it is passed along the flow path. If a cell has not ruptured it is returned, or kept in, the lysing chamber (312) where it may further be lysed until rupture occurs.

Each cell analysis device (200) also includes a number of encapsulation inlets (206) to deliver a quantity of an encapsulating fluid into the microfluidic channel (202) to encapsulate a lysate of each cell.

In this example, the cell analysis system (308) also includes a controller (316) to analyze the cell. The controller (316) includes various components to make such an analysis. First, the controller (316) includes a lysate analyzer (318) to analyze properties of a lysate of the cell. That is, after the cell has been ruptured, the contents therein may be analyzed and information provided to the lysate analyzer (318). A variety of pieces of information can be collected from the lysate. For example, cytoplasmic fluid within the cell may provide a picture of the current mechanisms occurring within the cell. Examples of such mechanisms include ribonucleic acid (RNA) translation into proteins, RNA regulating translation, and RNA protein regulation, among others. As another example, nucleic fluid can provide a picture of potential mechanisms that may occur within a cell, mechanisms such as mutations. In yet another example, mitochondrial fluid can provide information as to the origin of the cell and the organism's matrilineal line.

The controller also includes a rupture analyzer (320) which determines a rupture threshold of the cell based on the parameters of the agitation when the cell membrane ruptures. That is, as described above a cell may be exposed to one or multiple agitation cycles. The parameters of the different agitation cycles can be passed to the rupture analyzer (320) which analyzes parameters of an agitation when a cell membrane ruptures. The rupture analyzer (320) may use this information to perform a variety of analytical operations. That is, the rupture analyzer (320) can determine the rupture threshold by knowing how many agitation cycles the cell was exposed to and the intensity of each agitation cycle. Thus, the rupture analyzer (320) determines at what agitation intensity the cell ultimately ruptures. With such information on hand, the rupture analyzer (320) can determine certain properties of the cell including cell type, cell state, etc.

Thus, the present cell analysis system (308) provides a way to collect information related to both the lysate and the mechanical properties of the cell membrane from a single sample. Being able to collect both pieces from a single sample removes any bias resulting from intra-sample variation. Being able to collect both pieces of information from a single sample also makes more effective use of the sample. That is, rather than requiring two groups of the sample, one for mechanical testing and one for genetic testing, both pieces of information from one group of the sample.

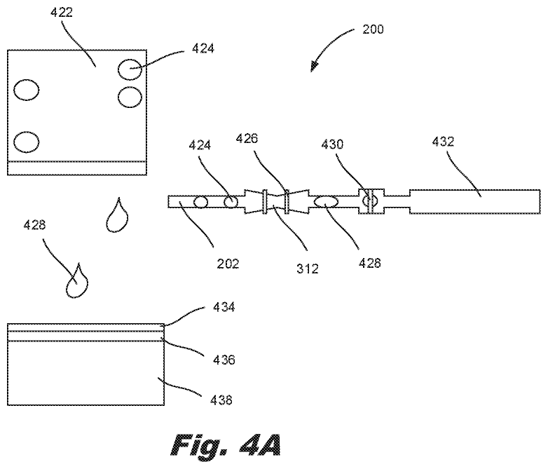

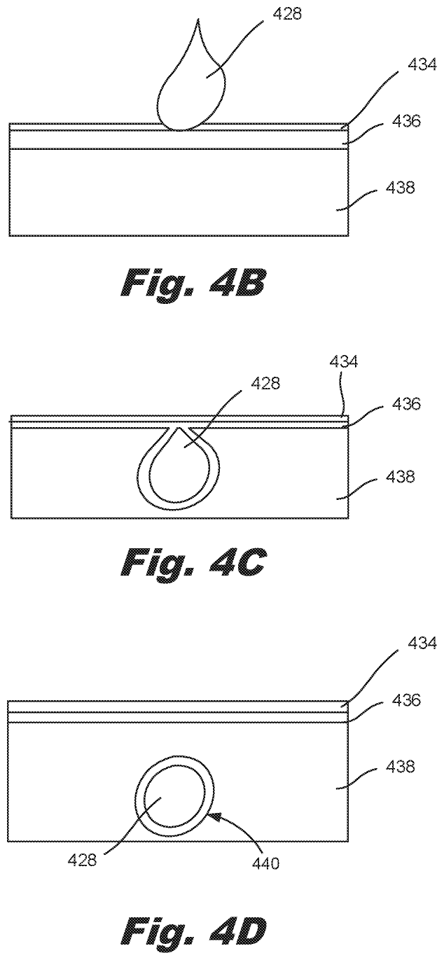

FIGS. 4A-4D are diagrams of a cell analysis device (200), according to another example of the principles described herein. In the example depicted in FIGS. 4A-4D, 1) transporting the lysate (428) of each cell (424) to a downstream analysis device includes ejecting the lysate (428) through an orifice and 2) encapsulating the lysate (428) of an individual cell includes ejecting the lysate (428) towards a well of a well plate that is filled, at least in part, with the encapsulating fluid. That is, in this example the encapsulation mechanism includes ejecting a lysate (428) onto a substrate that is prepared with an encapsulating fluid disposed on the top.

In this example, the cell analysis system (FIG. 3, 308) includes a cell reservoir (422) fluidically coupled upstream of each cell analysis device (200) to hold the volume of cells (424) that are to be analyzed. Cells are passed to each cell analysis device (200) in parallel even though each cell analysis device (200) may analyze a single cell at a time.

In the example depicted in FIG. 4A, the microfluidic cell analysis device(s) (200) are disposed under the cell reservoir (422). In this example, gravity, or a pressure draw from a pump, pulls the cells into the microfluidic cell analysis device (200). Specifically, cells (424) are passed along a microfluidic channel (202) into a lysing chamber (312) where a controlled lysis operation is executed based on feedback received by the sensors (426). Once lysed, the lysate (428) continues along a flow path until it reaches a firing chamber.

In the example depicted in FIG. 4A, the microfluidic cell analysis device (200) includes ejectors (430) to eject the lysate (428) to the intended surface, i.e., the well plate.

The ejector (430) may include a firing resistor or other thermal device, a piezoelectric element, or other mechanism for ejecting fluid from the firing chamber. For example, the ejector (430) may be a firing resistor. The firing resistor heats up in response to an applied voltage. As the firing resistor heats up, a portion of the fluid adjacent the firing resistor vaporizes to form a bubble. This bubble pushes the cell to be analyzed out an orifice and onto a surface such as a micro-well plate. As the vaporized fluid bubble collapses, a vacuum pressure along with capillary force draws additional fluid towards the ejector (430), and the process repeats. In this example, the ejector (430) may be a thermal inkjet ejector (430).

In another example, the ejector (430) may be a piezoelectric device. As a voltage is applied, the piezoelectric device changes shape which generates a pressure pulse that pushes a fluid out the orifice. In this example, the ejector (430) may be a piezoelectric inkjet ejector (430).

In some examples, the cell analysis device (200) also includes a waste reservoir (432) to collect waste fluid such as the carrier fluid in which the cell is disposed.

As described above, the encapsulating fluid may include two parts. An oil-based fluid (436) with a surfactant (434). The surfactant (434) may be a film disposed on top of the oil-based fluid (436). In some examples, a dispersant (438) may be disposed below the encapsulating fluid. In other examples, the dispersant (438) is not implemented and the lysate (428) is ejected into a well filled just with the encapsulating fluid, i.e., the surfactant (434) and the oil-based fluid (436).

The dispersant is an anti-coalescent. That is, the dispersant (438) prevents the individual slugs of encapsulated lysate from agglomerating. Specifically, a dispersant molecule coats the particle (or droplet) to be dispersed by aligning itself with one end towards the molecule and the other to the continuous phase. The part of the molecule that faces the continuous phase repulses the same part of molecules from another particle for example by electrostatic repulsion, thus preventing the particles from coalescing. Without such a dispersant (438) individual slugs of encapsulated lysate may bind together. As one specific example, the dispersant (438) may be a cationic detergent such as cetyl trimethyl ammonium bromide (CTAB) or a sodium-based sulfate such as sodium dodecyl sulfate (SDS). Other examples of dispersants include sodium dodecyl benzene sulfonate, dimethyl ether of tetradecyl phosphonic acid, lauryl mono-ethanol, N-dodecyl pyridinium chloride and abietic acid. In other examples, the dispersant (438) may be any chemical to prevent the coalescence of the oil-based encapsulating fluid. In one specific example, the dispersant (438) may be water.

FIGS. 4B-4D depict the lysate (428) at various stages of ejection into a well plate that includes a surfactant (434) film and oil-based fluid (436) that make up the encapsulating fluid and the dispersant (438) on which it is formed. Specifically, FIG. 4B depicts the lysate (428) as it enters the encapsulating fluid. As depicted in FIG. 4C as the lysate (428) drops through the encapsulating fluid, the surfactant (434) enables a bond between the aqueous lysate (438) and the oil-based fluid (436). Once past the encapsulating fluid as depicted in FIG. 4D, the lysate (428) is completely enveloped as an encapsulated lysate (440) slug. As described above, while FIGS. 4A-4D depict ejection of a lysate (428) into a well plate that includes an encapsulating oil-based fluid as well as a dispersant (438), in some examples, the ejection may be into just the encapsulating fluid.

FIG. 5 is a flow chart of a method (500) of cell encapsulation, according to another example of the principles described herein. According to the method (500), serially fed cells (FIG. 4A, 424) are received (block 501) and individually lysed (block 502) in a lysing chamber (FIG. 3, 312). These operations may be performed as described above in connection with FIG. 1.

In this example, the method (500) includes adding (block 503) a densification agent to the cell (FIG. 4A, 424). That is, in some examples, it may be desirable to have the lysate (FIG. 4A, 428) sink to the bottom of the well plate more quickly than it otherwise would. In so doing, a scientist may not need to wait as long to do subsequent analysis. Accordingly, a densification agent may be added (block 503). The densification agent may be any agent that increases the overall average density of the droplet. In one specific example, the densification agent includes metallic particles that are mixed with the lysate (FIG. 4A, 428) to increase the density of the aqueous lysate solution such that it sinks within the dispersant (FIG. 4A, 438) and does not rise to the surface where the encapsulating oil (FIG. 4A, 436) is positioned. While FIG. 5 depicts the addition (block 503) of the densification agent to the lysate (FIG. 4A, 428) following lysis in some examples the addition (block 503) may occur at other times. For example, the densification agent may be added (block 503) prior to lysis.

Regardless of when the densification agent is added (block 503), the lysate (FIG. 4A, 428) may be ejected (block 504) through the orifice and encapsulated (block 505) via the ejection towards a well plate that is filled, at least in part, with the encapsulating fluid.

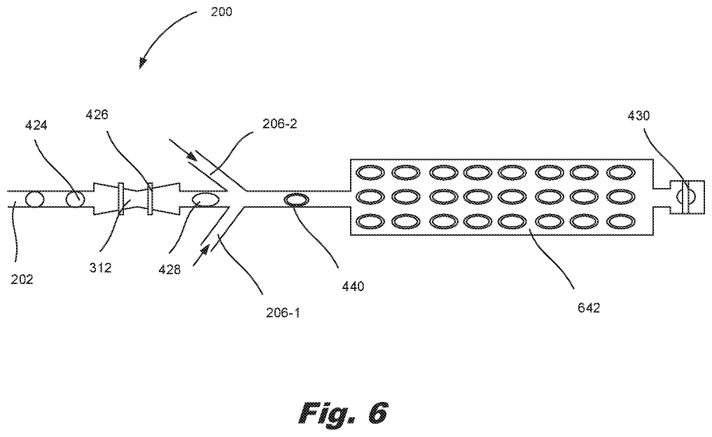

FIG. 6 is a diagram of a cell analysis device (200), according to another example of the principles described herein. In the example depicted in FIG. 6, rather than expelling the lysate (428) unencapsulated through a film of encapsulating fluid, the cell analysis device (200) includes integrated encapsulation inlets (206-1, 206-2) that pump encapsulating fluid into the microfluidic channel (202). That is, cells (424) pass through the microfluidic channel (202) into the lysis chamber (312) where they are lysed. The lysate (428) then continues through the microfluidic channel (202). At this time the encapsulating fluid (which includes the surfactant (FIG. 4A, 434) and an oil-based fluid (FIG. 4A, 436) such as hexa-methyl di-siloxane) is injected into the stream. The nature of the amphiphilic surfactant (FIG. 4A, 434) bonds the encapsulating oil around the lysate (428) to form encapsulated lysate (440) slugs that are isolated from the lysates (428) of other cells. Thus, the lysate (428) of individual cells can be separately analyzed without having been contaminated, or otherwise mixed with the carrier fluid and/or lysate of other cells.

In the example depicted in FIG. 6, encapsulated lysate (440) slugs are stored in a storage chamber (642). The storage chamber (642) holds a quantity of encapsulated lysate (440) slugs prior to their ejection or transport to a downstream analysis system.

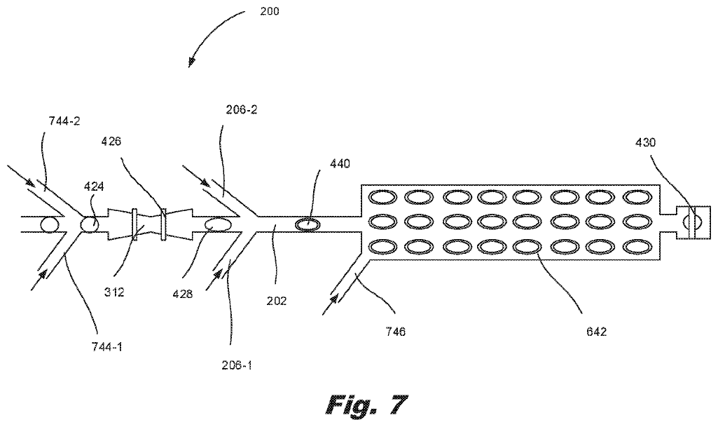

FIG. 7 is a diagram of a cell analysis device (200), according to another example of the principles described herein. FIG. 7 depicts an example wherein the cell analysis device (200) includes the microfluidic channel (202) that delivers a cell (424) to a lysis chamber (312) where it is to be lysed. Following lysis, the lysate (428) is mixed with an encapsulating fluid injected by the encapsulation inlets (206-1, 206-2) forming encapsulated lysate (440) slugs which are held in a storage chamber (642) prior to ejection by the ejector (430).

In this example however, the encapsulation inlets (206-1, 206-2) rather than injecting an oil-based fluid (FIG. 4A, 436)/surfactant (FIG. 4A, 434) mixture, inject just the oil-based fluid (FIG. 4A, 436). The surfactant (FIG. 4A, 434) is injected separately. Accordingly, the cell analysis device (200) includes a number of surfactant inlets (744-1, 744-2) to deliver a surfactant (FIG. 4A, 434) to the microfluidic channel (202). Specifically, in the example depicted in FIG. 7, the surfactant inlets (744-1, 744-2) are upstream of the lysis device (FIG. 1, 104) along the flow path.

FIG. 7 also depicts an example where the cell analysis device includes a number of secondary encapsulation inlets (746) to deliver a secondary encapsulating fluid to envelope an encapsulated lysate (440) slug. For example, the second encapsulating fluid may be delivered into the storage chamber (642) where the encapsulated lysate (440) slugs are stored. In this example, the encapsulating fluid may be oil-based and the secondary encapsulating fluid may be aqueous, with or without a surfactant mixed in. In other words, a water (lysate (428)) in oil (encapsulating fluid) in water (secondary encapsulating fluid) emulsion may be held within the storage chamber (642). In this example, the oil-based encapsulating fluid may be a variety of fluids including a silicon-based oil. An example of a silicon-based encapsulating fluid is polyphenyl methylsiloxane. In other examples, the oil-based encapsulating fluid includes a hydrocarbon oil. An example of a hydrocarbon-based oil is a composition that includes a mixture of hydrocarbons and C13-14 isoparaffin.

The second encapsulating fluid, which may be an aqueous buffer may be a phosphate buffered saline, which may have the following ingredients NaCl 137 mM, KCl 2.7 mM, Na2HPO4 10 mM, and KH2PO4 1.8 mM.

Such a solution provides even greater assurance that the lysate (428) does not mix with other lysates (428). In this example, the doubly-encapsulated lysate (428) slugs may be broken down downstream for further lysate analysis.

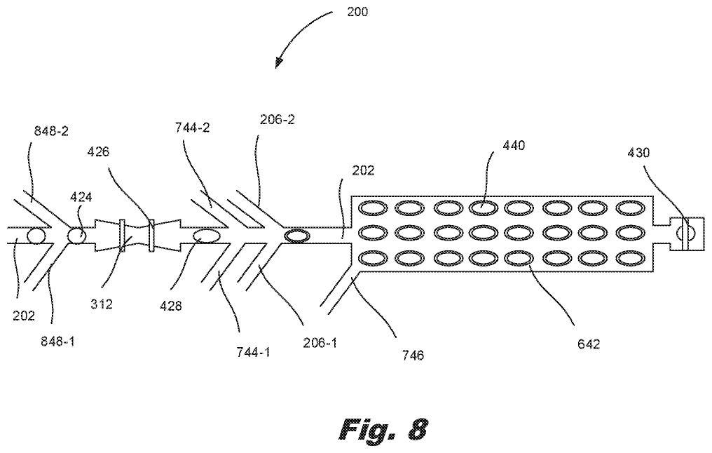

FIG. 8 is a diagram of a cell analysis device (200), according to another example of the principles described herein. FIG. 8 depicts an example wherein the cell analysis device (200) includes the microfluidic channel (202) that delivers a cell (424) to a lysis chamber (312) where it is to be lysed. Following lysis, the lysate (428) is mixed with an encapsulating fluid injected by the encapsulation inlets (206-1, 206-2) forming encapsulated lysate (440) slugs which are held in a storage chamber (642) prior to ejection by the ejector (430).

In the example depicted in FIG. 8, the surfactant inlets (744) are downstream of the lysis device (FIG. 1, 104) along the flow path. Doing so may preserve the cell. That is, in some scenarios, the surfactant may lyse the cell (424). Accordingly, placing the surfactant inlets (744) downstream of the lysis device (FIG. 1, 104) ensures that the addition of the surfactant does not alter the cell in undesired ways.

FIG. 8 also depicts a number of densification inlets (848-1, 484-2) to deliver a densification agent to the microfluidic channel (202). As described above, the densification agent increases the overall density of a cell and lysate, thus reducing the time between ejection by the ejector (430) and when the cell can be analyzed by a downstream analysis device. Note that while FIG. 8 depicts the densification agent being added prior to lysing, in some examples, such as described in connection with FIG. 5, the densification agent may be added following lysis.

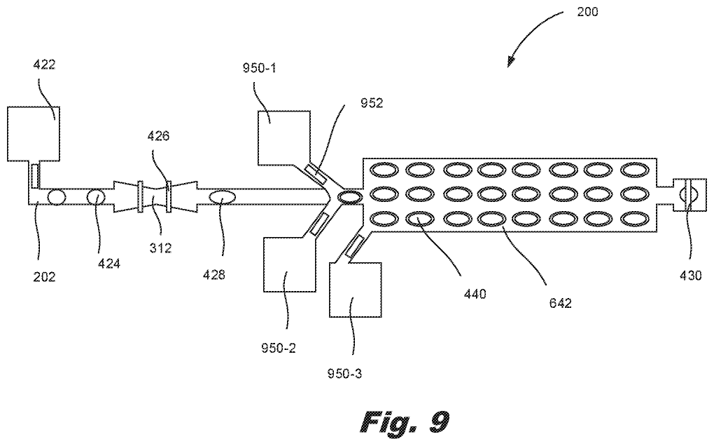

FIG. 9 is a diagram of a cell analysis device (200), according to another example of the principles described herein. FIG. 9 depicts an example wherein the cell analysis device (200) includes the microfluidic channel (202) that delivers a cell (424) to a lysis chamber (312) where it is to be lysed. Following lysis, the lysate (428) is mixed with an encapsulating fluid forming encapsulated lysate (440) slugs which are held in a storage chamber (642) prior to ejection by the ejector (430).

In the example depicted in FIG. 9, in addition to components previously described, the cell analysis device (200) includes a number of fluid sources (422, 950-1, 950-2, 950-3). Each source contains at least one of the sample, the encapsulating fluid, the secondary encapsulating fluid, and the surfactant. Specifically, in the example depicted in FIG. 9, the encapsulating fluid is contained in a first fluid source (950-1) and a second fluid source (950-2) and the second encapsulating fluid is contained in a third fluid source (950-3). Note that while FIG. 9 depicts a few fluid sources, additional fluid sources may be present for example to hold the surfactant and the densification agent described in previous examples. In other words, the example depicted in FIG. 9 describes a cell analysis device (200) wherein the fluid inlets as well as the sources from where the fluid originates, are formed on/in the same substrate as the lysis device (FIG. 1, 104) and the encapsulation mechanism.

To draw fluid from the different sources (950), each inlet may include a pump (952). For simplicity, one pump (952) is indicated with a reference number. The pumps (952) may be integrated into walls of the respective inlets. In some examples, the pumps (952) may be inertial pumps which refers to a pump (952) which is in an asymmetric position within the inlet. The asymmetric positioning within the inlet facilitates an asymmetric response of the fluid to the pump (952). The asymmetric response results in fluid displacement when the pump (952) is actuated. In some examples, the pumps (952) may be thermal inkjet resistors, piezo-drive membranes or any other displacement device.

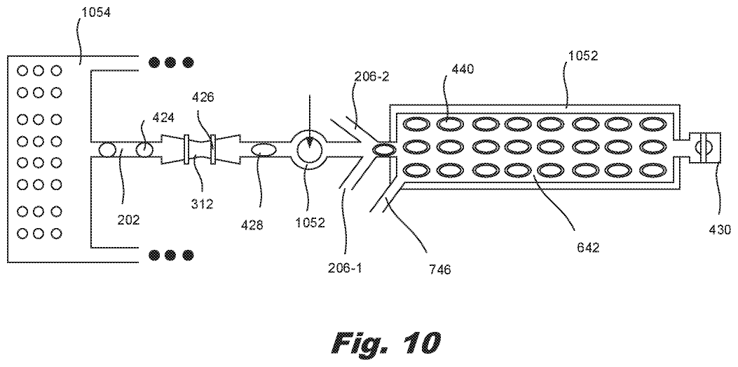

FIG. 10 is a diagram of multiple cell analysis devices (200), according to another example of the principles described herein. FIG. 10 depicts the cells (424), microfluidic channel (202), lysis chamber (312), sensors (426), lysate (428), encapsulation inlets (206), secondary encapsulation inlet (746), encapsulated lysate (440) slugs, storage chamber (642), and ejector (430) described previously.

In the example depicted in FIG. 10, the cell analysis device (200) also includes a cell sorter (1054) to pass different cells to be analyzed to different cell analysis devices (200). That is, as described above, the cell analysis system (FIG. 3, 308) may include different cell analysis devices (200) such that parallel cell analysis operations may be carried out. In some examples, the different cell analysis devices (200) operate on the same cells, in other examples different cell analysis devices (200) operate on different cells. Accordingly, the cell sorter (1054) separates different cells from one another.

In one example, the cell sorter (1054) includes an array of columns spaced apart to facilitate separation of cellular particles from the surrounding fluid based on a size of the cellular particles. In this particular example, cellular particles are sorted by directing the sample, which includes the cellular particles, through an input channel and into a separation chamber. The separation chamber includes an array of columns, or posts that are spaced apart so as to direct cellular particles in the sample along different flow paths based on the size of the particles.

In some examples, the columns may be arranged in a particular arrangement, such as one that coincides with deterministic lateral displacement (DLD). DLD uses a specific arrangement of obstacles such as the columns to control the path of particles to separate particles larger than a threshold diameter from those smaller than the threshold diameter through collisions with the obstacles. That is, in a flow, when a particle is larger than the threshold diameter, its center is positioned such that collision with an obstacle causes the larger particle to flow to one side of the obstacle, while collision of objects smaller than the threshold diameter with the same obstacle causing the smaller particles to flow to the other side of the obstacle. Accordingly, particles of different sizes are directed to different output channels.

While FIG. 10 depicts the columns as having a particular shape and size in a particular configuration, the columns may be formed having any cross-sectional shape and size and arranged in any configuration. Moreover, while FIG. 10 depicts a particular type of sorting mechanism, other types of separating mechanisms may be used to separate cellular particles form the surrounding fluid in which they are dispersed.

In addition to encapsulation and lysis, the cell analysis devices (200) may include components for other operations. For example, the cell analysis device (200) may include a chamber (1052) wherein reagents may be added. For example, a reagent is added to the microfluidic channel (202) through an orifice in the chamber (1052). Examples of reagents that may be added include a master mix, library prep reagents, and DNA preservation reagents. In some examples, the reagent added is a chemical cell lysis reagent.

In the example depicted in FIG. 10, the cell analysis device (200) further includes a resistor plane (1052) for heating the storage chamber (642). Such a resistor plane allows for the cyclic heating of the encapsulated lysate (440) slugs. Such an operation may be referred to as thermo-cycling and is an operation executed in many cell analysis operations such as digital drop PCR and sequence amplification operations.

In summary, such a cell analysis device, system, and method 1) allow single cell analysis of a sample; 2) allow combined cell analysis, i.e., a genetic analysis and a mechanical property analysis; 3) can be integrated onto a lab-on-a-chip; 4) are scalable and can be parallelized for high throughput, 5) are low cost and effective; 6) are automated; 7) preserve lysate content from contamination by other lysates and other components such as protein and debris which may be in the carrier fluid and RNAase and DNAase which may have been used to lyse the cell; 8) reduce dilution of lysate contents thus improving downstream analysis; and 9) reduce the effect of potential user error. However, the devices disclosed herein may address other matters and deficiencies in a number of technical areas.

What is claimed is:

1. A method, comprising:
receiving, in a microfluidic channel, serially fed cells to be encapsulated;
individually lysing each cell;
transporting a lysate of each cell to a downstream analysis device; and
encapsulating the lysate of an individual cell with an encapsulating fluid.

2. The method of claim 1, wherein:
transporting the lysate of each cell to a downstream analysis device comprises ejecting the lysate through an orifice; and
encapsulating the lysate of an individual cell comprises ejecting the lysate towards a well of a well plate that is filled, at least in part, with the encapsulating fluid.

3. The method of claim 1, wherein the encapsulating fluid comprises:
an oil that is immiscible with the lysate; and
surfactant mixed with the oil.

4. The method of claim 1, further comprising adding a densification agent to the cell prior to lysis, wherein the densification agent increases an overall density of the cell and encapsulating fluid mixture.

5. The method of claim 1, further comprising adding a densification agent to the lysate following lysis, wherein the densification agent increases an overall density of the lysate and encapsulating fluid mixture.

6. A cell analysis device, comprising:
a microfluidic channel to serially transport cells along a flow path;
a lysis device along the microfluidic channel to rupture a cell membrane of a cell; and
a number of encapsulation inlets to deliver a quantity of an encapsulating fluid into the microfluidic channel to encapsulate a lysate of the cell.

7. The cell analysis device of claim 6, further comprising at least one of:
a number of secondary encapsulation inlets to deliver a secondary encapsulating fluid to envelope an encapsulated lysate; and
a number of surfactant inlets to deliver a surfactant to the microfluidic channel.

8. The cell analysis device of claim 7, wherein the surfactant inlets are upstream of the lysis device along the flow path.

9. The cell analysis device of claim 7, wherein the surfactant inlets are downstream of the lysis device along the flow path.

10. The cell analysis device of claim 7, wherein:
the encapsulating fluid is oil-based; and
the secondary encapsulating fluid is aqueous.

11. The cell analysis device of claim 7, further comprising a number of fluid sources, each source to contain at least one of the encapsulating fluid, the secondary encapsulating fluid, and the surfactant.

12. A cell analysis system, comprising:
at least one cell analysis device, each cell analysis device comprising:
a microfluidic channel to serially feed individual cells from a volume of cells into a lysing chamber;
at least one feedback-controlled lysing element in the lysing chamber to agitate a cell; and
a number of encapsulation inlets to deliver a quantity of an encapsulating fluid into the microfluidic channel to encapsulate a lysate of each cell; and
a controller to analyze the cell, the controller comprising:
a lysate analyzer to analyze properties of a lysate of the cell; and
a rupture analyzer to analyze parameters of an agitation when a cell membrane ruptures.

13. The cell analysis system of claim 12, further comprising a storage chamber to hold encapsulated lysate.

14. The cell analysis system of claim 12, further comprising a cell sorter to pass different cells to be analyzed to different cell analysis devices.

15. The cell analysis system of claim 12, further comprising a number of densification inlets to deliver a densification agent to the microfluidic channel.
